(19) United States
(12) Patent Application Publication
(10) Pub. No.: US 2023/0157635 A1
(43) Pub. Date: May 25, 2023

(54) MULTIMODAL BIOMETRIC HUMAN MACHINE INTERFACE HEADSET

(71) Applicant: Dhiraj JEYANANDARAJAN, Irvine, CA (US)

(72) Inventor: Dhiraj JEYANANDARAJAN, Irvine, CA (US)

(21) Appl. No.: 18/058,253

(22) Filed: Nov. 22, 2022

Related U.S. Application Data
(60) Provisional application No. 63/283,192, filed on Nov. 24, 2021.

(51) Int. Cl. A61B 5/00; A61B 5/0205; A61B 5/374

(52) U.S. Cl. CPC A61B 5/6803; A61B 5/0205; A61B 5/374; A61B 5/02416

(57) ABSTRACT

A headset and methods for using the headset to collect EEG data, PPG data and IMU data are disclosed. Raw EEG data generated from the headset (or other device) may be received. The EEG data may be run through spectral analysis to isolate various spectral components in each channel, isolating the brain wave components for each channel. Similar data for heart rate, respiratory rate and heart rate variability can be extrapolated from PPG data as well as the positional movements in space along with acceleration and angular velocity may be determined from IMU data. A visual display may be generated based on the isolated components.

### CROSS REFERENCE TO RELATED APPLICATION(S)

This application claims priority to U.S. Provisional Pat. Application Serial No. 63/283,192, filed on Nov. 24, 2021, which is hereby incorporated herein by reference in its entirety.

### FIELD OF THE INVENTION

The present invention relates generally to a headset and noninvasive sensors for real-time measuring of electroencephalography (EEG), photoplethysmography (PPG), and inertial measurement unit (IMU) signals from locations on the forehead and head in the detection of brain activity.

### BACKGROUND OF THE INVENTION

Brain waves can be detected via EEG, which involves monitoring and recording electrical impulse activity of the brain, typically noninvasively. EEG data can be generated by placing a number of electrodes (often part of a brain-computer interface (BCI) headset) on or near the subject’s scalp. The electrodes detect the electrical impulses generated by the brain and send signals to a computer that records the results. The data from each electrode may be deemed a channel representing data from a portion of the brain where the electrode is located. Each channel may have an active and reference electrode in a montage used in the differential amplification of the source signal.

Brain waves may be detected as a time-varying signal and comprise components having different spectral characteristics such as frequency and power. As an example, brain waves may include the following brain wave components: delta waves, theta waves, alpha waves, beta waves, and gamma waves. The spectral characteristics of the brain waves may indicate different mental states based on factors such as source location, duration, coherence and dominance or amplitude.

### SUMMARY OF THE INVENTION

Traditional systems in the field of non-invasive plethysmography (e.g., using an instrument to measure changes in volume within an organ of a body) have been implementing improvised calculations of PPG signals in order to measure changes in volume of human organs. For example, one method of signal quality analysis uses the perfusion index (PI) to measure a peripheral perfusion or ratio of pulsatile blood flow to non-pulsatile static blood flow in a patient’s peripheral tissue. Another method of signal quality analysis uses skewness to measure the probability distribution of a real valued random variable about its mean value. Another method of signal quality analysis uses regression techniques to map the values of these parameters to PPG signal classifications.

However each of these traditional methods have limitations. For example, PI works best for signals that are received through transmissive type PPG sensors and not for reflective type PPG. In some examples, PI is not comparable with reflective type signals and traditional SQI may be limited due to the reflective type sensor. The signal may be classified based on SNR and standard deviation values combined, for example, the SNR calculation equal to 20 * log10 (Mean / standard deviation of signal) dB.

Various embodiments of the present disclosure may include capacitive non-dermal EEG electrodes mounted within the headset device for brain wave signal detection and data acquisition. The headset can enable real-time measuring of EEG as well as PPG signals from locations of the user’s head in the detection of brain activity.

The headset may be embodied in a durable polymer. The headset may also include one or more adjustable slider arms on either side of the headset (e.g., for a better fit of the headset to the user’s head via, for example, extending or retracting across the user’s forehead), LED lights on either side (e.g., to identify power, charging, or programmable function indicators), one or more replaceable EEG sensors (e.g., with surfaces coated in silver, silver chloride, or conductive polymer), one or more PPG sensors (e.g., on the forehead), and/or a charging port. A single frontal piece may be employed in addition to or instead of the slider arms. Other components of the headset may be included as well, including a microphone, adhesive, and/or connectors between components.

The slider arms can be placed on either side of the headset and may be adjustable. The slider arms may extend or retract for positioning on the forehead of the user. The single frontal piece extending across the user’s forehead may optionally be employed connecting the slider arms or in place of the slider arms.

The LED lights can be placed on either side of the headset or at other locations of the headset to indicate power, charging status, any connections to a host device, or other programmable functions.

The EEG sensors can detect electricity generated by a user’s brain as brain waves and generate data. The data corresponding with the brain waves can be filtered, amplified, analyzed, and/or recorded (e.g., in a wave pattern).

The PPG sensors can determine the PPG data. The PPG sensors may be placed along the headset to correspond with forehead locations when the user is wearing the headset. The sensor placement may identify one or more optimal locations for determining PPG signals.

In some examples, the headset may apply automatic gain control (AGC) and signal to noise ratio (SNR) calculations of the PPG signals. The AGC may compensate for different skin tones using positive or negative feedback loops from standard values. In some examples, the signal strength of PPG recorded is highly dependent on skin tone. Since the skin tissues lie on the optical path of PPG, darker skin tones absorb large amounts of 660 nm wavelength — red light, whereas the lighter / pale skin tones reflect back most of the light causing amplifier saturation. To ensure optimum signal quality through different demographics, AGC may be applied to keep the PPG signal amplitude under or above desired threshold value.

The SNR is also determined. In some examples, the PPG signals may be highly sensitive to sensor and skin movement. When a reflective type sensor is implemented with the headset, light may be traveling toward the artery or blood vessel and may also be traveling from the artery or blood vessel as reflected light. This slight change in optical path between the light traveling towards and reflected from the user’s skin (e.g., based on sensor movement or skin movement) can cause a disturbance in recorded PPG signal. When the sensor is not placed properly or there is a motion artifact, the SNR value may decrease and standard deviation of that data recording may increase. This change indication may be provided to the user (e.g., via a software application or graphical user interface) to adjust the PPG sensor location or fix the PPG sensor to properly record the values.

Other noise reduction methods may be implemented other than SNR. For example, due to the nature of implementing an indirect measurement process, wearable devices may inevitably face challenges caused by baseline drift and Motion Artifacts (MAs), especially during exercise and under free living conditions.

Although the classical Adaptive Threshold Peak Detection (ATPD) algorithm is capable of resolving baseline drift in PPG signal analysis by detection of peak positions in the time domain, ATPD may be vulnerable to MAs. Adaptive Noise Cancellation (ANC) has the ability to reduced unwanted MAs by introducing multi-sensor accelerometer and gyroscope signals and it is being widely used for cancelling MAs and noise in PPG signals. However, the ANC algorithm fails if the MAs have a close enough main frequency component to the heartbeat rate in the PPG signal. Adaptive noise cancellation algorithm utilizes Discrete cosine transform and Hilbert transform calculation over complete frequency range (i.e. 0 to 50 Hz) for every second of data of PPG signal. While implementing this, there may be a lag in data acquisition due to high computational complexity of the algorithm. To overcome these problems, a Discrete cosine transform and Hilbert transform calculation may be computed over complete frequency range (i.e. 0 to 8.53 Hz or 512 data points) for every second of data of PPG signal and instead of processing 0 to 50 Hz which corresponds to 3000 data points of DCT values. Real-time data acquisition (with no lag) of PPG may be obtained.

Once the optimal value of PPG is captured, AGC may set the LED current. Once the LED current is set, the AGC can calculate SNR and standard deviation to estimate signal quality of the signal from the site of the recording (e.g., fixed to the headset).

As illustrative examples, the LED current range can be correlated for various skin tones. For example, for light brown skin tones, the LED current range (Red) may be 8 to 9 mA and the LED current range (IR) may be 6. In another example, for moderate brown skin tones, the LED current range (Red) may be 12 to 16 mA and the LED current range (IR) may be 6. In another example, for dark to deep dark skin tones, the LED current range (Red) may be 20 to 30 mA and the LED current range (IR) may be 6.

One or more capacitive non-dermal EEG electrodes may be incorporated with the headset. Each of the electrodes may have an elongated shape (e.g., rectangle or ellipse) or a non-elongated shape (e.g., square or circle), and/or a curved shape (e.g., a curvature to match a natural curvature of a surface of a head). The electrodes’ surfaces may be coated in gold, silver, silver chloride, or other conductive polymer. The texture of the electrodes may be smooth or any other texture that can increase a surface area of the electrode.

In some examples, the electrodes collect data using noninvasive, electrical brain signal measurements absent the use of an interface material between the electrode and the skin (e.g., an electrolyte, in a EEG gel or paste form, etc.). The user may not need to add gel or saline solution to improve signal quality of the capacitive non-dermal contact electrodes.

Each electrode may be coupled with foam, spring, gel-containing material, or other support that can provide some pressure from the headset to the electrode, in order to help apply pressure to the electrode to remain in contact with the hair or head of the user. The pressure can help ensure a better fit and help conform the electrode (and headset overall) to the curvature of portions of the head and/or irregular (or regular) bumps on a surface of the head.

The electrodes may be placed at locations of the headset based on general head anthropometry and experimental trials. The electrodes may be curved. The curvature of the electrodes may vary based on the placement of the electrode location. Different head parts may correspond with different curvatures. In some examples, eight electrodes may be placed around the headset in various designs and utilitarian functions for capacitive non-dermal EEG electrodes.

The electrodes may be replaceable. For example, the headset may have one or more ports to plug in an electrode into headset. When an electrode becomes inoperable, the electrode may be unplugged from the port and replaced with a new electrode for easy replacement.

The charging port can provide power to the components of the headset and optional wired connectivity for transmitting data. In a rechargeable scenario, a cable may be removably coupled with the charging port in the headset and a power outlet to provide electrical connectivity while charging the headset. In some examples, the cable may remain attached to the charging port in the headset and a power outlet during operation of the headset (e.g., in research and gaming environments where even millisecond delays have significance).

The headset may be used to collect data using an IMU (also referred to herein as an IMU sensor) within the headset as a control system. The IMU may include an accelerometer, gyroscope, feature extraction, sensor fusion, and other components or engines for generating data. The data can be stored, inferred, or retrieved from a memory incorporated with the headset as well. In some examples, the data may be transmitted to a computer system for processing and analysis.

The computer system may receive the data from the headset and implement controls based on the data. As an illustrative example, the headset may be moved along an X and Y axis and, on a corresponding graphical user interface provided at a display coupled with the computer system, an object can be moved in accordance with the headset movement. In other words, the headset may be used to control the object using head motion as an alternative human-computer interface device.

In some examples, the computer system may improve the data using a spectrum noise cancellation process to remove motion artifacts (e.g., generated by the EEG or PPG sensors). The computer system may execute spectrum denoising to help reduce the noise in the data. As an illustrative example, the denoised data may be used to estimate heart rate and respiration rate of the user in a non-invasive form.

These and other objects, features, and characteristics of the system and/or method disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

The drawings are provided for purposes of illustration only and merely depict typical or example implementations. These drawings are provided to facilitate the reader’s understanding and shall not be considered limiting of the breadth, scope, or applicability of the disclosure. For clarity and ease of illustration, these drawings are not necessarily drawn to scale.

FIG. 1 provides a left-perspective view of an embodiment of the headset, in accordance with one or more implementations of the invention.

FIG. 2 provides a right-perspective view of an embodiment of the headset, in accordance with one or more implementations of the invention.

FIGS. 3-4 provide illustrative views of the headset as worn by a user, in accordance with one or more implementations of the invention.

FIG. 5 provides an illustrative view of a slider arm of the headset, in accordance with one or more implementations of the invention.

FIG. 6 provides an illustrative view of PPG and EEG sensors of the headset, in accordance with one or more implementations of the invention.

FIG. 7 provides a projection view of an EEG electrode of the headset, in accordance with one or more implementations of the invention.

FIG. 8 provides an isometric view of an EEG electrode of the headset, in accordance with one or more implementations of the invention.

FIG. 9 provides an exploded view of an EEG electrode of the headset, in accordance with one or more implementations of the invention.

FIG. 10 provides an illustrative placement of a set of EEG electrodes in a first layout of the headset, in accordance with one or more implementations of the invention.

FIGS. 11-14 provide illustrative embodiments electrodes of the headset, in accordance with one or more implementations of the invention.

FIG. 15 provides an illustrative placement of a set of EEG electrodes in a second layout of the headset, in accordance with one or more implementations of the invention.

FIGS. 16-19 provide illustrative embodiments electrodes of the headset, in accordance with one or more implementations of the invention.

FIG. 20 provides an illustrative placement of a set of EEG electrodes in a third layout of the headset, in accordance with one or more implementations of the invention.

FIGS. 21-24 provide illustrative embodiments electrodes of the headset, in accordance with one or more implementations of the invention.

FIG. 25 provides an illustrative process for replacing electrodes of the headset, in accordance with one or more implementations of the invention.

FIG. 26 provides an illustrative example of generating inertial data, in accordance with one or more implementations of the invention.

FIG. 27 provides an illustrative example of generating accelerometer data, in accordance with one or more implementations of the invention.

FIG. 28 provides an illustrative example of generating gyroscope data, in accordance with one or more implementations of the invention.

FIG. 29 illustrates 3D angular mapping using various components of the headset, in accordance with one or more implementations of the invention.

FIG. 30 illustrates the user’s head movement for generating data, in accordance with one or more implementations of the invention.

FIG. 31 illustrates a process of converting data to interactions with a desktop display, in accordance with one or more implementations of the invention.

FIG. 32 illustrates an example of a process of calculating a heart rate, in accordance with one or more implementations of the invention.

FIG. 33 illustrates an example of a process of calculating a heart rate and/or respiration rate, in accordance with one or more implementations of the invention.

FIG. 34 illustrates an example of a process of determining LED current, in accordance with one or more implementations of the invention.

FIG. 35 illustrates an example of a process of determining a good or improper signal, in accordance with one or more implementations of the invention.

FIG. 36 provides a left-perspective view of an embodiment of the headset, in accordance with one or more implementations of the invention.

### DETAILED DESCRIPTION OF THE DRAWINGS

It is to be understood that the figures and descriptions of the present invention may have been simplified to illustrate elements that are relevant for a clear understanding of the present invention, while eliminating, for purposes of clarity, other elements found in a typical headset or typical method of using a headset. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, structures, apparatuses, systems, methods, features, and advantages will be or become apparent to one with skill in the art upon examining the drawings and the detailed description herein. It is intended that all such additional devices, structures, apparatuses, systems, methods, features, and advantages be protected by the accompanying claims.

For purposes of this disclosure, the term “headset” is interchangeable with the terms helmet, cap, hat, lid, wrap, band, and/or any combination thereof, or other head covering.

The invention described herein relates to a headset comprising a curved frame; a plurality of capacitive non-dermal EEG sensors connected with the curved frame; one or more PPG sensors connected with the forehead portion of the curved frame, wherein locations and curves of the plurality of EEG sensors are formatted in accordance with a head shape of a user; and one or more adjustable slider arms on either side of the headset.

The invention described herein also relates to systems and methods for measuring of PPG signals from a forehead location and capacitive non-dermal EEG sensors distributed over the surface of the head in the detection of brain activity having one or more physical processors programmed with computer program instructions that, when executed by the one or more physical processors, cause the computer system to perform the method, the method comprising: receiving, by one or more sensors, raw EEG data; applying spectral analysis to one or more channels of the raw EEG data to isolate spectral components in the channels; and using this data to create data outputs based on the spectral analysis that may be either displayed in raw form or utilized in algorithms to determine mental states of the user.

It will be appreciated by those having skill in the art that the implementations described herein may be practiced without these specific details or with an equivalent arrangement. In various instances, well-known structures and devices are shown in block diagram form to avoid unnecessarily obscuring the implementations.

### Example Headset Architecture

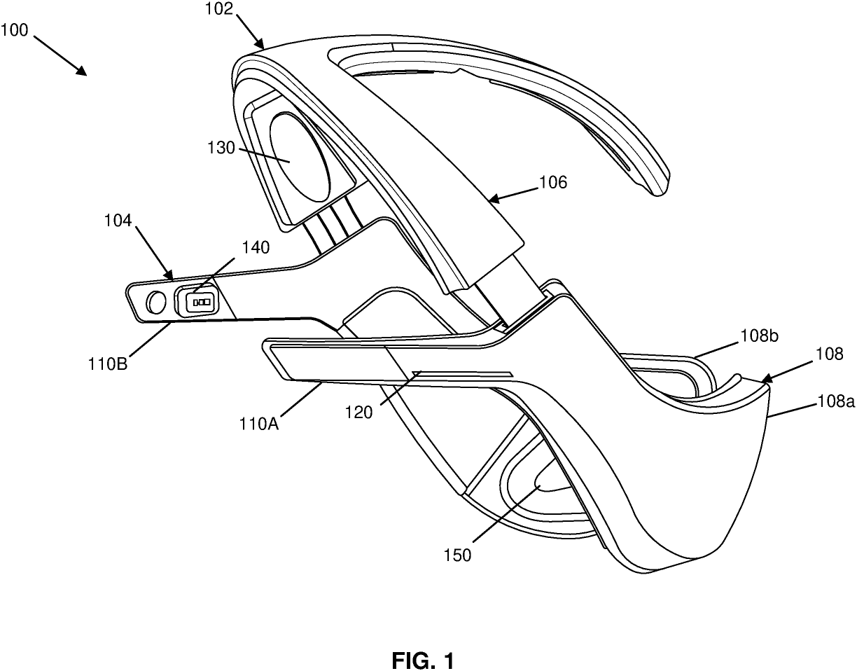

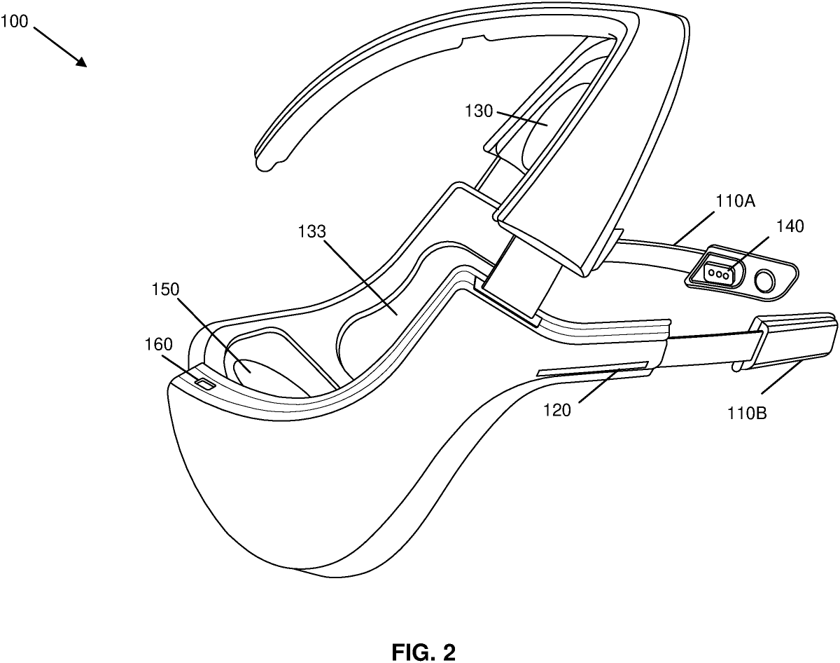

FIGS. 1-2 provide a left-perspective view and a right-perspective view of an embodiment of the headset, respectively, in accordance with one or more implementations of the invention. In these figures, an embodiment of headset 100 is provided. Headset 100 may be a non-dermal contact headset device for brain wave signal detection and data acquisition. Headset 100 can enable real-time measuring of PPG signals from forehead location of the user’s head in the detection of brain activity, and can be employed absent the use of traditional methods like the perfusion index, skewness, and regression techniques.

Headset 100 may be embodied in a durable polymer or other flexible or non-flexible material. Headset 100 may also include a frontal piece including one or more adjustable slider arms 110, including left slider arm 110A and right slider arm 110B, on either side of headset 100 (e.g., for a better fit of the headset to the user’s head), LED lights 120 on either side (e.g., to identify power, charging, or programmable function indicators), one or more replaceable EEG sensors 130 (e.g., with surfaces coated in silver, silver chloride, or conductive polymer), reference electrode 133 (for the EEG sensors 130), one or more PPG sensors 140 (e.g., on the forehead), and a charging port 160. Other components of headset 100 may be included as well, including a mic, adhesive, and/or connectors between components.

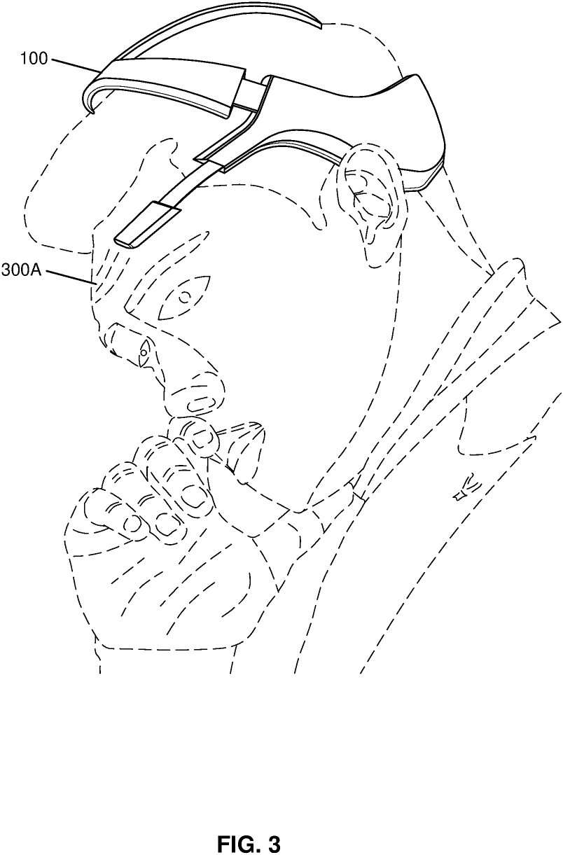

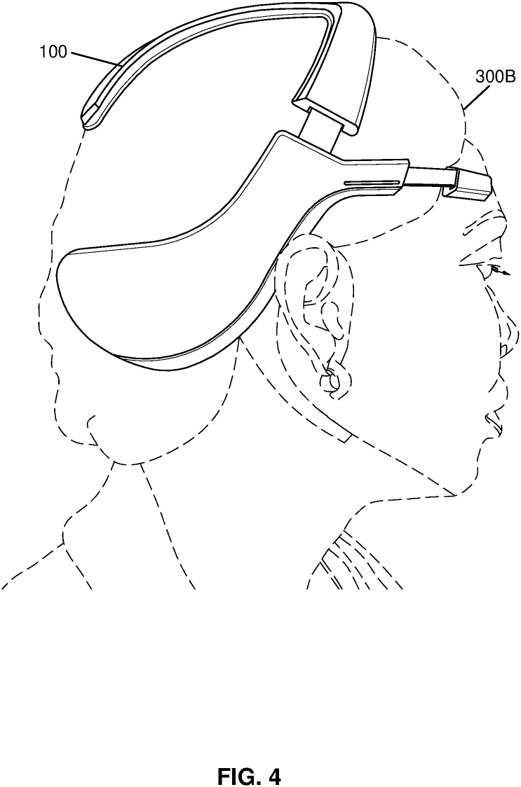

Additional views of headset 100 are illustrated in FIGS. 3-4. While headset 100 is worn, EEG signals are determined from the capacitive non-dermal EEG electrodes, illustrated as first user 300A and second user 300B. The EEG signals correspond with brain activity of each user.

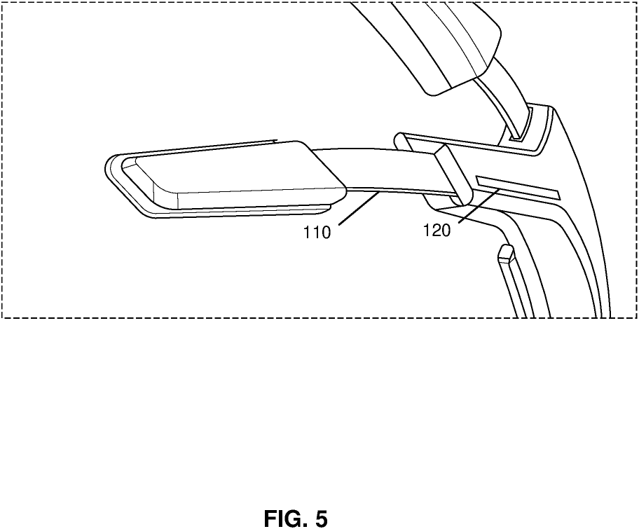

Adjustable slider arm 110 and LED light 120 are illustrated in FIG. 5. For example, adjustable slider arm 110 can be placed on either side of headset 100 and may be adjustable/movable (see, for example, slider arms 110a, 110b in FIGS. 1-2). Adjustable slider arm 110 may extend or retract for positioning on the forehead of the user. Additionally, LED lights 120 can be placed on either side of headset 100 or at other locations of the headset 100, to indicate changes with the devices, including increasing or decreasing power, active or deactive charging status, initiating or dropping connections to a host device, or other programmable functions.

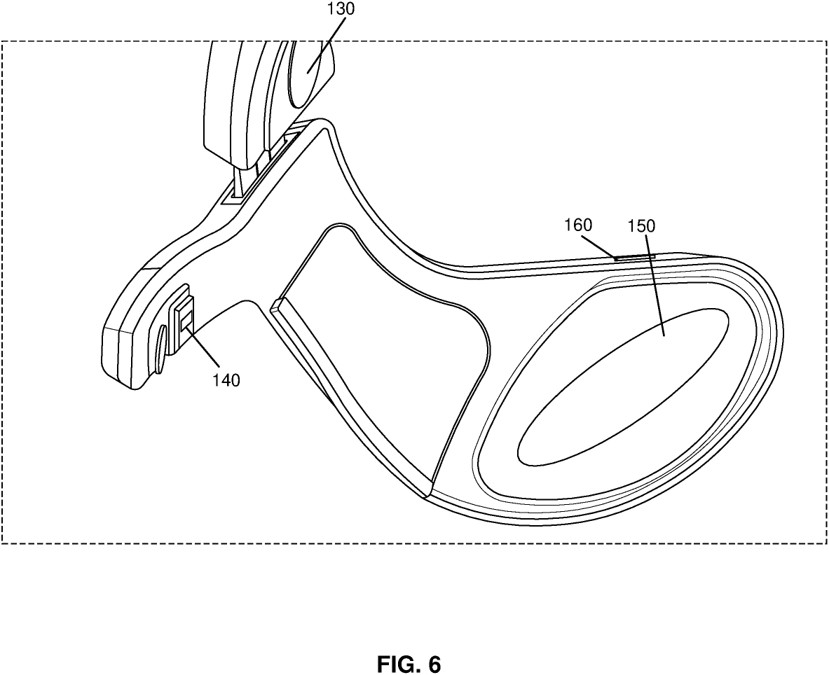

One of the EEG sensors 130 and one of the PPG sensors 140 are illustrated in FIG. 6. The EEG sensors 130 can detect electricity generated by a user’s brain as brain waves and generate data. More or fewer EEG sensors may be provided with headset 100. The data corresponding with the brain waves can be processed (e.g., filtered, amplified, analyzed, and/or recorded (e.g., in a wave pattern)).

PPG sensors 140 can determine the PPG data. PPG sensors 140 may be placed along headset 100 to correspond with forehead location when the user is wearing the headset.

The PPG data may be altered. In some examples, headset 100 may apply automatic gain control (AGC) and signal to noise ratio (SNR) calculations of the PPG signals. The AGC may compensate for different skin tones using positive or negative feedback loops from standard values. In some examples, the signal strength of PPG recorded is highly dependent on skin tone. Since the skin tissues lie on the optical path of PPG, darker skin tones absorb large amounts of 660 nm wavelength — red light, whereas the lighter / pale skin tones reflect back most of the light causing amplifier saturation. To ensure optimum signal quality through different demographics, AGC may be applied to keep the PPG signal amplitude under or above desired threshold value.

The SNR may also be determined. In some examples, the PPG signals generated by PPG sensors 140 may be highly sensitive to sensor and skin movement. When a reflective type sensor is implemented with headset 100, light may be traveling toward the artery or blood vessel and may also be traveling from the artery or blood vessel as reflected light. This slight change in optical path between the light traveling towards and reflected from the user’s skin (e.g., based on sensor movement or skin movement) can cause a disturbance in recorded PPG signal. When the sensor is not placed properly or there is a motion artifact, the SNR value may decrease and standard deviation of that data recording may increase. This change indication may be provided to the user (e.g., via a software application or graphical user interface) to adjust the PPG sensor location or fix the PPG sensor to properly record the values.

Other noise reduction methods may be implemented other than SNR. For example, due to the nature of implementing an indirect measurement process, wearable devices may inevitably face challenges caused by baseline drift and Motion Artifacts (MAs), especially during exercise and under free living conditions.

Although the classical Adaptive Threshold Peak Detection (ATPD) algorithm is capable of resolving baseline drift in PPG signal analysis by detection of peak positions in the time domain, ATPD may be vulnerable to Mas. Adaptive Noise Cancellation (ANC) has the ability to reduced unwanted Mas by introducing multi-sensor accelerometer and gyroscope signals and it is being widely used for cancelling Mas and noise in PPG signals. However, the ANC algorithm fails if the Mas have a close enough main frequency component to the heartbeat rate in the PPG signal. Adaptive noise cancellation algorithm utilizes Discrete cosine transform and Hilbert transform calculation over complete frequency range (i.e. 0 to 50 Hz) for every second of data of PPG signal. While implementing this, there may be a lag in data acquisition due to high computational complexity of the algorithm. To overcome these problems, a Discrete cosine transform and Hilbert transform calculation may be computed over complete frequency range (i.e. 0 to 8.53 Hz or 512 data points) for every second of data of PPG signal and instead of processing 0 to 50 Hz which corresponds to 3000 data points of DCT values. Real-time data acquisition (with no lag) of PPG may be obtained.

Once the optimal value of PPG is captured, AGC may set the LED current. Once the LED current is set, the AGC can calculate SNR and standard deviation to estimate signal quality of the signal from the site of the recording (e.g., fixed to headset 100).

As illustrative examples, the LED current range can be correlated for various skin tones. For example, for light brown skin tones, the LED current range (Red) may be 8 to 9 mA and the LED current range (IR) may be 6. In another example, for moderate brown skin tones, the LED current range (Red) may be 12 to 16 mA and the LED current range (IR) may be 6. In another example, for dark to deep dark skin tones, the LED current range (Red) may be 20 to 30 mA and the LED current range (IR) may be 6.

Charging port 160 can provide power to the components of the headset and optional wired connectivity. A cable may be removably coupled with the charging port in the headset and a power outlet to provide electrical connectivity to the headset. In some examples, the cable may remain attached to headset 100 during operation of the headset (e.g., in research and gaming environments where even millisecond delays have significance).

Embodiments are directed to a headset 100 including a curved frame 102 (e.g., FIG. 1) including: a frontal curved frame portion 104 configured to be worn on a forehead portion of a head of a user; and an upper curved frame portion 106 configured to be worn on an upper head portion of the head of the user. The headset 100 also includes: one or more PPG sensors 140 coupled to the frontal curved frame portion 104; One or more (capacitive non-dermal) EEG sensors (and a bias sensor/electrode) are coupled to the frontal curved frame portion 104 and/or upper curved frame portion 106. One or more additional capacitive non-dermal EEG sensors 150 (also referred to throughout the disclosure as electrodes 150) may be optionally coupled to the posterior curved frame portion 108.

In an embodiment, the one or more additional EEG sensors are coupled to only the upper curved frame portion.

In an embodiment, each of the PPG sensors 140 includes a curved outer surface, wherein the coupling location of each PPG sensor to the front curved frame portion 104 and a shape of the curved outer surface of each PPG sensor are configured to correspond with a corresponding location and curvature of the forehead or temporal portion of the head of the user.

In an embodiment, each of the EEG sensors 130 includes a curved outer surface, wherein the coupling location of each EEG sensor 130 to the frontal curved frame portion 104 and/or upper curved frame portion 106 and a shape of the curved outer surface of each EEG sensor 130 are configured to correspond with a corresponding location and curvature of the upper head portion of the head of the user.

In an embodiment, each of the EEG sensors 130 is a capacitive non-dermal contact type EEG sensor that is configured to be positioned either in direct contact with skin or be positioned over hair (i.e., not in direct contact with skin) on the head of the user, when the headset 100 is worn by the user.

In an embodiment, the frontal curved frame portion 104 includes two length-adjustable slider arms 110A, 110B that are retractable and extendable from opposite side portions of the curved frame.

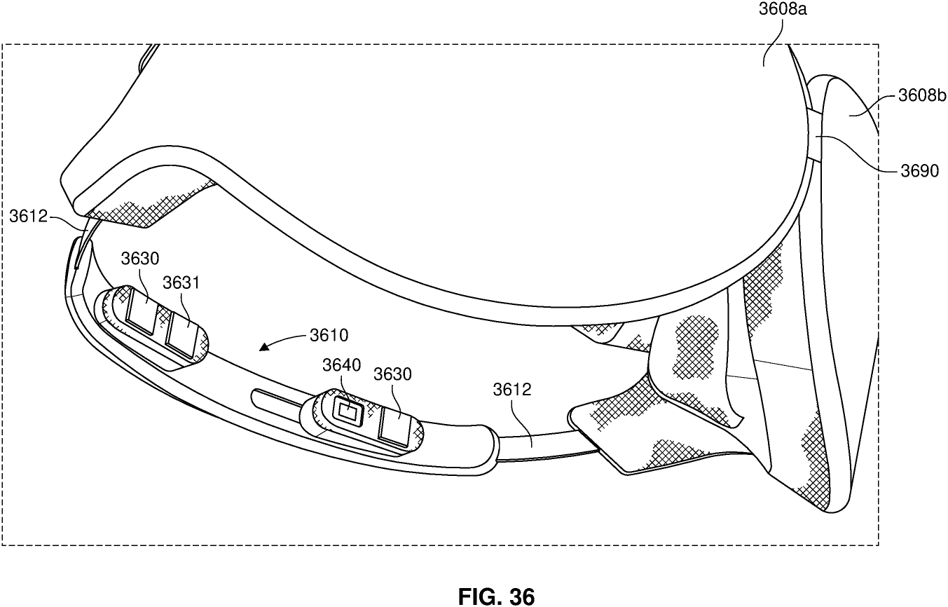

In an embodiment, the frontal curved frame portion 104 includes an adjustable single frontal piece 3610 (see FIG. 36 which is described more fully below) coupled between opposite side portions of the curved frame 102, wherein a PPG sensor 3640 and EEG sensors 3630 are positioned along the single frontal piece 3610. The single frontal piece 3610 may be adjustable with respect to the opposite side portions of the curved frame via rigid or flexible retractable/extendable slider arms 3612 or, alternatively, via elastic arms (not shown).

In an embodiment, the curved frame 102 further includes a posterior curved frame portion 108. One or more additional EEG sensors 150 are coupled to the posterior curved frame portion 108.

In an embodiment, the curved frame 102 further includes a posterior curved frame portion 108 including two posterior parts 108a, 108b. The headset may further include one or more electrodes 150 coupled to at least one of the two posterior parts 108a, 108b. As shown in FIG. 36, the corresponding two posterior parts 3608a, 3608b may be coupled together via an elastic member 3690.

In an embodiment, each of the EEG sensors 130 (again, for example, FIG. 1) may be removable or replaceable.

In an embodiment, the headset 100 further includes a charging port 160.

In an embodiment, the headset 100 further includes one or more LED lights 120 indicating power, charging status, and/or connection to a host device.

In an embodiment, the headset 100 further includes an IMU sensor.

### Electrode Embodiments

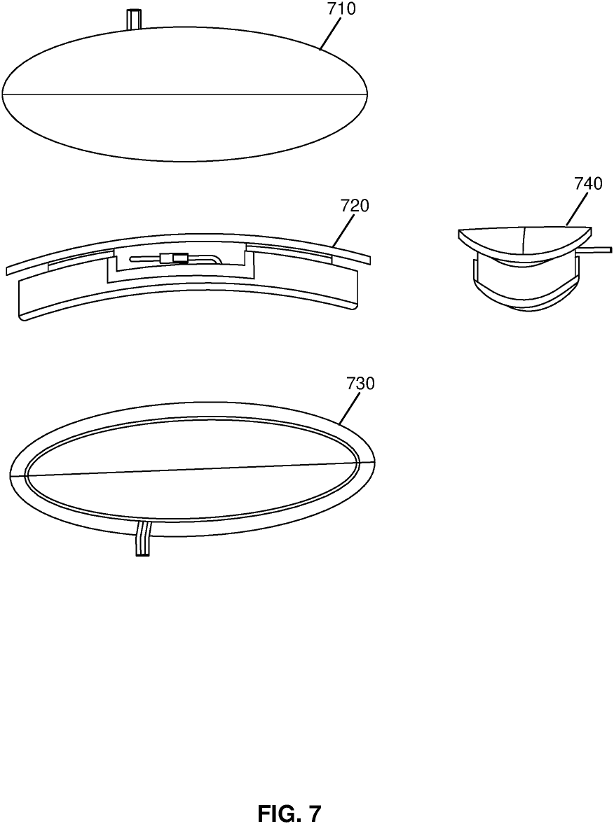

Various embodiments of electrode construction, placement, and design are described throughout the disclosure. For example, FIGS. 7-9 illustrates various views of an electrode design, in accordance with one or more implementations of the invention. One or more electrodes 150 may be removably attached to headset 100 (and may be replaceable) and configured to receive brain waves.

In the projection view of an electrode design shown in FIG. 7, electrode top 710, electrode middle 720, electrode bottom 730, and electrode side 740 are provided for illustrative purposes and should not be limiting to the disclosure.

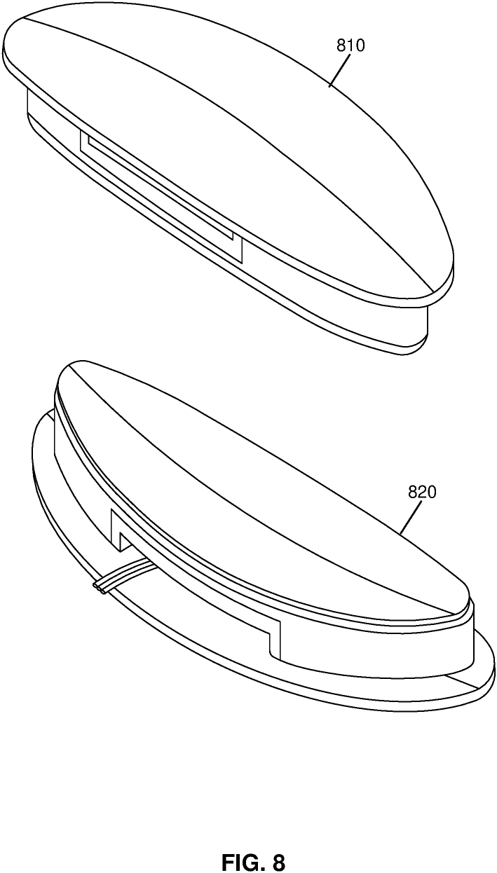

In the isometric view of an electrode design shown in FIG. 8, electrode top 810 and electrode bottom 820 are provided for illustrative purposes and should not be limiting to the disclosure.

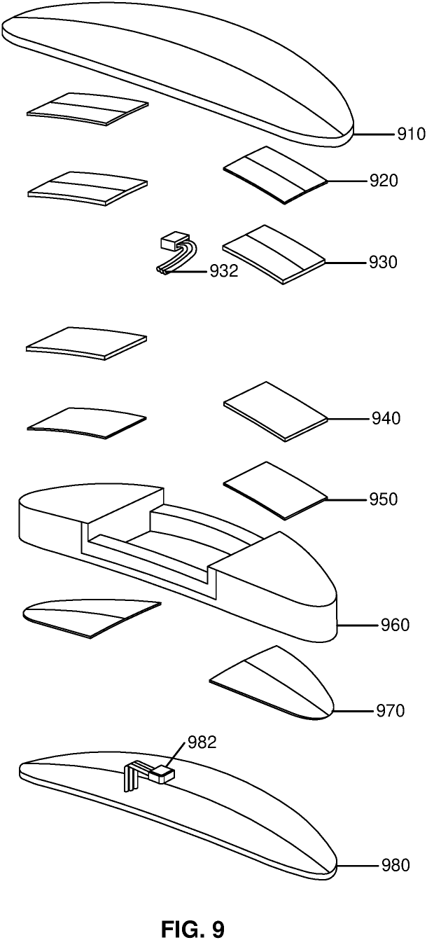

In the exploded view of an electrode design shown in FIG. 9, electrode top 910, first adhesive 920, first side of fastener 930, first side of connector 932, second side of fastener 940, second adhesive 950, foam 960, third adhesive 970, electrode 980, and second side of connector 982. These and other components of the exploded view of an electrode design are provided for illustrative purposes and should not be limiting to the disclosure. For example, foam 960 can provide some pressure from the headset to the electrode 910, in order to help apply pressure to electrode 980 to remain in contact with the hair or head of the user. The pressure can help ensure a better fit and help conform electrode 980 (and headset 100 overall) to the curvature of portions of the head and/or irregular (or regular) bumps on a surface of the head.

Each of the electrodes 150 may have an elongated shape (e.g., rectangle or ellipse) or a non-elongated shape (e.g., square or circle), and/or a curved shape (e.g., a curvature to match a natural curvature of a surface of a head). The surfaces of electrodes 150 may be coated in, for example, gold, silver, silver chloride, or other conductive polymer. The texture of electrodes 150 may be smooth or any other texture that can increase a surface area of electrode 150.

The configuration of each electrode 150 may form a low density (e.g., 2-channel system) to a high density (e.g., 256-channel system) array. For example, headset 100 may comprise a 8-channel EEG system with an active and reference electrode. In some implementations, each electrode 150 may correspond to a specific channel input of the scanner. For example, first electrode 150A may correspond to a first channel, second electrode 150B may correspond to a second channel, and so on. In some embodiments, each channel may have an active and reference electrode in a montage used in the differential amplification of the source signal.

The channels of each electrode may be configured to receive different components, for example such as delta, theta, alpha, beta, and/or gamma signals, each of which may correspond to a given frequency range. In a non-limiting example implementation, delta waves may correspond to signals between 0 and 3.5 Hz, theta waves may correspond to signals between 3.5 and 8 Hz, alpha waves may correspond to signals between 8 and 12 Hz, beta waves may correspond to signals between 12 and 30 Hz, and gamma waves may correspond to signals above 30 Hz. These example frequency ranges are not intended to be limiting and are to be considered exemplary only.

In some implementations, electrodes 150 may be attached at locations spread out across headset 100. Electrodes 150 may be configured to detect electric potentials generated by the brain from the low ionic current given off by the firing of synapses and neural impulses traveling within neurons in the brain. These electric potentials may repeat or be synchronized at different spectral characteristics such as frequency and power according to the previously listed brain wave types (e.g. alpha and beta). These spectral characteristics of the brain waves may be separated from the single superimposed frequency signal detected at each electrode 150. In various implementations, this isolation, separation, decomposition, or deconstruction of the signal is performed via spectral analysis.

In various implementations, headset 100 may be configured to receive raw EEG data generated by one or more electrodes 150. In some implementations, headset 100 may be configured to perform initial signal processing on the detected brain waves. For example, headset 100 may be configured to run the raw EEG data through a high and low bandpass filter prior to the filtered data being run through a fast Fourier transform (FFT) to isolate the spectral frequencies of each channel. Each channel may be run through a high and low bandpass filter. In some implementations, headset 100 may be configured to perform error detection, correction, signal decomposition, signal recombination, and other signal analysis. Accordingly, headset 100 may be configured to filter, analyze, and/or otherwise process the signals captured by one or more electrodes 150.

In some examples of the “10-20 international system” of electrode placement, Channel 1 may correspond to the Fp1 location and Channel 2 may correspond to Fp2. The active electrode may be placed along the frontal curved portion and the reference electrode may be placed on the temporal region. As described herein, filtered data for each channel may be run through spectral analysis to isolate the spectral frequencies of each channel. The power of the theta (e.g., 4-7 Hz), alpha (e.g., 8-12 Hz), beta (e.g., 13-20 Hz), and gamma (e.g., 21-50 Hz) components of each channel for a given sampled timeframe (e.g., 3 seconds) may be determined. The power of each of the isolated components may be used to generate a numerical output of the data that may be used for graphical visualization or incorporated into algorithms for brain-related measurements.

In some examples, electrodes 150 may collect data using noninvasive, electrical brain signal measurements absent the use of an interface material between electrode 150 and the skin (e.g., an electrolyte, in a EEG gel or paste form, etc.). The user may not need to add gel or saline solution to improve signal quality of the capacitive non-dermal contact electrodes 150.

Each electrode 150 may be coupled with foam, spring, gel-containing material, or other support that can provide some pressure from the headset to the electrode 150, in order to help apply pressure to electrode 150 to remain in contact with the hair or head of the user. The pressure can help ensure a better fit and help conform electrode 150 (and headset 100 overall) to the curvature of portions of the head and/or irregular (or regular) bumps on a surface of the head.

Electrodes 150 may be placed at various locations of headset 100 based on general head anthropometry and experimental trials. Electrodes 150 may be curved. The curvature of electrodes 150 may vary based on the placement of the electrode location. Different head parts may correspond with different curvatures. In some examples, eight electrodes may be placed around the headset in various designs and utilitarian functions for non-dermal EEG systems.

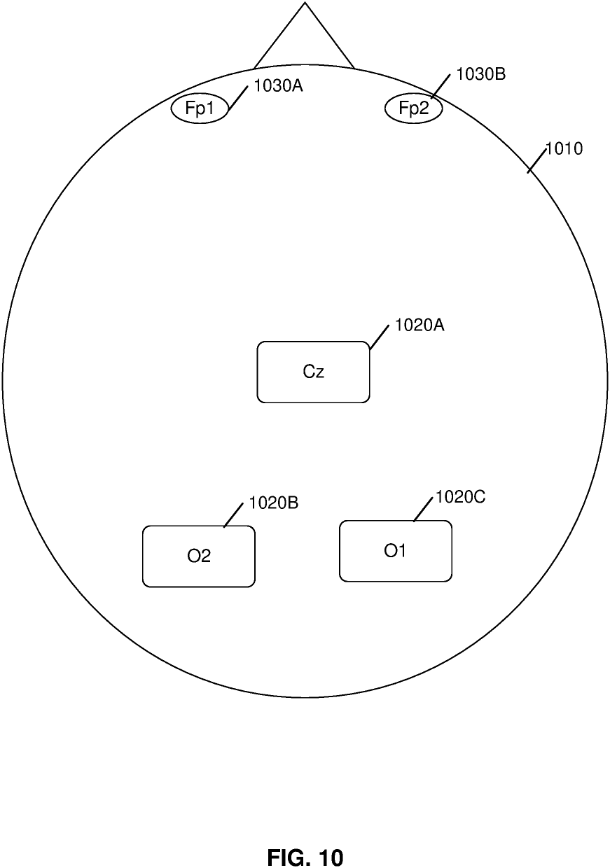

FIG. 10 illustrates a first layout of a plurality of electrodes and a curvature of the headset, in accordance with one or more implementations of the invention. In this illustration, a user’s head 1010 is provided relative to placement of one or more electrodes 1020, illustrated as first electrode 1020A, second electrode 1020B, and third electrode 1020C, and Fp1/Fp2 location electrodes 1030.

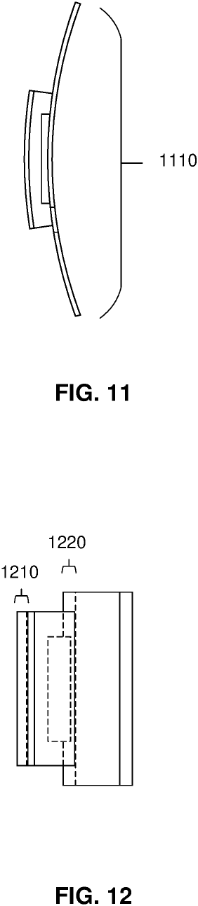

FIG. 11 illustrates a top view of an electrode in the first layout of FIG. 10. For example, the height 1110 of the electrode may be around 49 mm with a curvature of around 20° and a focal length of around 71 mm.

FIG. 12 illustrates a side view of an electrode in the first layout of FIG. 10. For example, the initial depth 1210 of the electrode may be around 0.5 mm and the secondary depth may be around 0.8 mm.

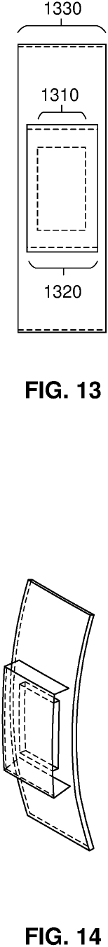

FIG. 13 illustrates a back view of an electrode in the first layout of FIG. 10. For example, the width of the innermost portion 1310 of the electrode may be around 8 mm, the width of the secondary portion 1320 of the electrode may be around 12 mm, and the width of the outermost portion 1330 of the electrode may be around 15 mm.

FIG. 14 illustrates a projected view of an electrode in the first layout of FIG. 10. In some examples, the height of the electrode is 50 mm and the width of the electrode is 15 mm.

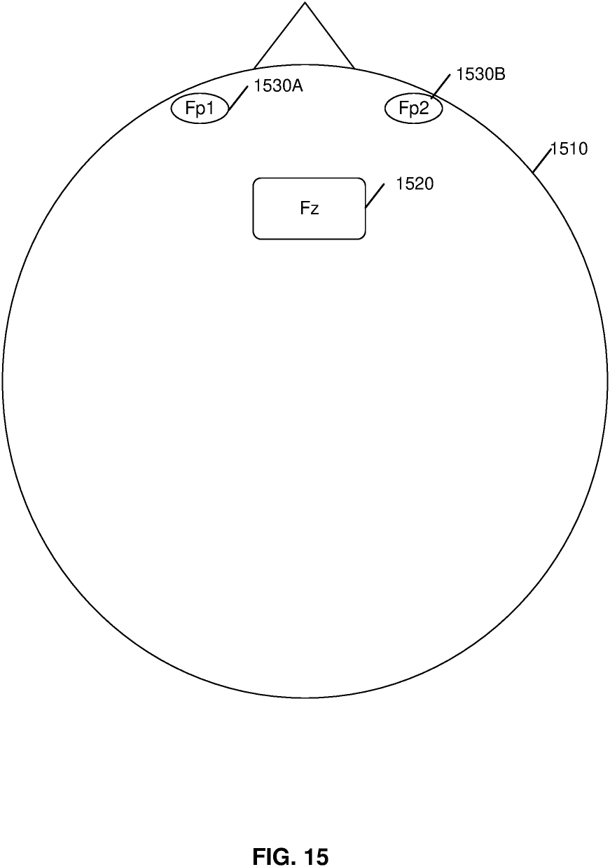

FIG. 15 illustrates a second layout of a plurality of electrodes and a curvature of the headset, in accordance with one or more implementations of the invention. In this illustration, a user’s head 1510 is provided relative to placement of one or more electrodes 1520, and Fp1/Fp2 location electrodes 1530.

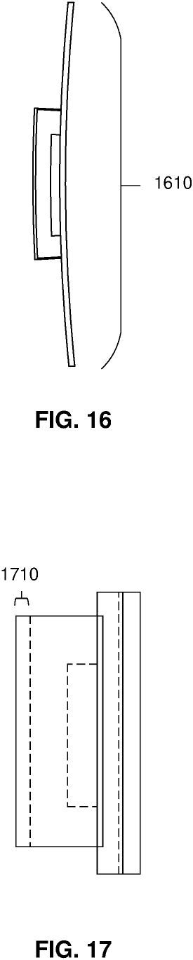

FIG. 16 illustrates a top view of an electrode in the second layout of FIG. 15. For example, the height 1610 of the electrode may be around 50 mm with a curvature of around 12°.

FIG. 17 illustrates a side view of an electrode in the second layout of FIG. 15. For example, the depth 1710 of the electrode may be around 0.5 mm.

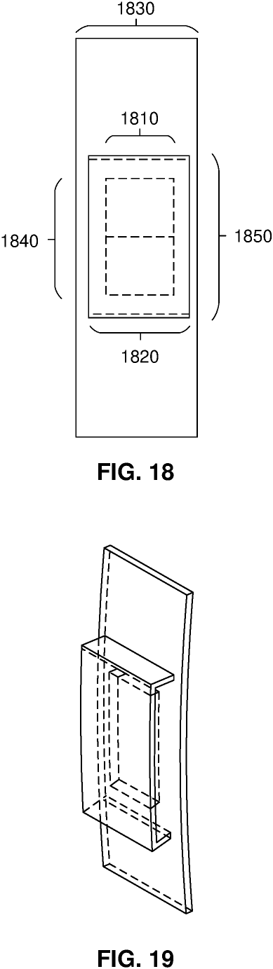

FIG. 18 illustrates a back view of an electrode in the second layout of FIG. 15. For example, the width of the innermost portion 1810 of the electrode may be around 8 mm, the width of the secondary portion 1820 of the electrode may be around 12 mm, and the width of the outermost portion 1830 of the electrode may be around 15 mm. The height of the innermost portion 1840 may be around 14 mm and the height of the secondary portion 1850 may be around 20 mm.

FIG. 19 illustrates a projected view of an electrode in the second layout of FIG. 15. In some examples, the height of the electrode is 50 mm and the width of the electrode is 15 mm.

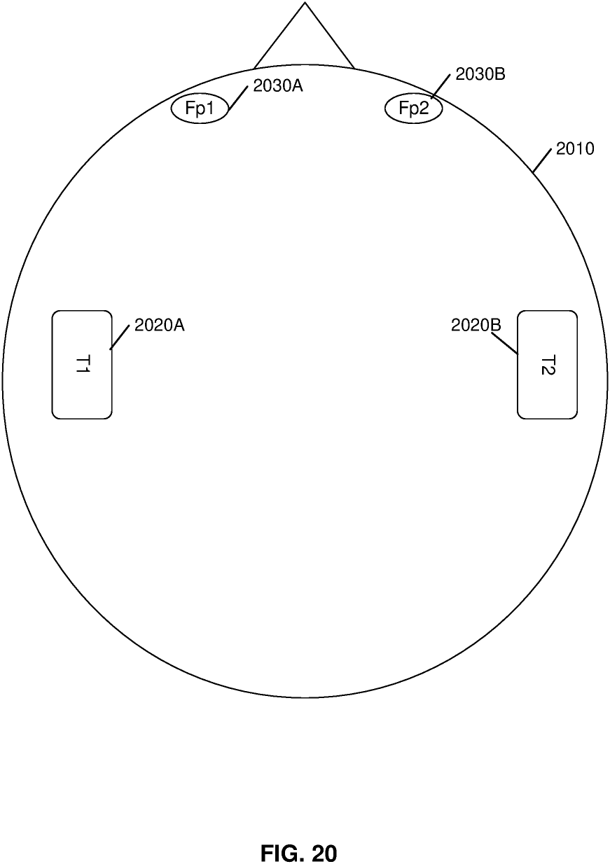

FIG. 20 illustrates a third layout of a plurality of electrodes and a curvature of the headset, in accordance with one or more implementations of the invention. In this illustration, a user’s head 2010 is provided relative to placement of one or more electrodes 2020, illustrated as first electrode 2020A and second electrode 2020B, and Fp1/Fp2 location electrodes 2030.

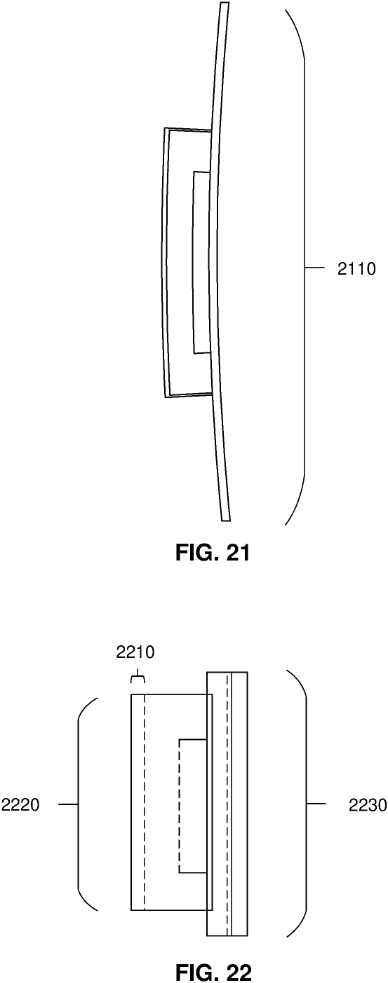

FIG. 21 illustrates a top view of an electrode in the third layout of FIG. 20. For example, the height 2110 of the electrode may be around 40 mm with a curvature of around 12 °.

FIG. 22 illustrates a side view of an electrode in the third layout of FIG. 20. For example, the depth 2210 of the electrode may be around 0.5 mm. The width of the innermost portion 2220 of the electrode may be around 10 mm and the width of the outermost portion 2230 of the electrode may be around 12 mm.

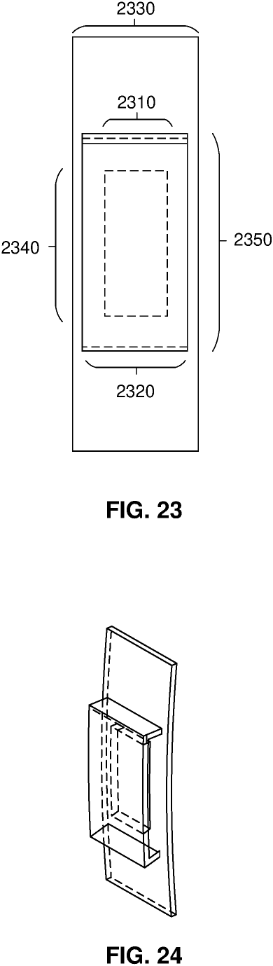

FIG. 23 illustrates a back view of an electrode in the third layout of FIG. 20. For example, the width of the innermost portion 2310 of the electrode may be around 6 mm, the width of the secondary portion 2320 of the electrode may be around 10 mm, and the width of the outermost portion 2330 of the electrode may be around 12 mm. The height of the innermost portion 2340 may be around 14 mm and the height of the secondary portion 2350 may be around 20 mm.

FIG. 24 illustrates a projected view of an electrode in the third layout of FIG. 20. In some examples, the height of the electrode is 40 mm and the width of the electrode is 12 mm.

In any of the embodiments described throughout the disclosure, including the embodiments illustrated in FIGS. 10-24, electrodes 150 may be replaceable. For example, headset 100 may have one or more ports to plug in an electrode into headset. When an electrode becomes inoperable, the electrode may be unplugged from the port and replaced with a new electrode for easy replacement.

Electrodes 150 may have an elongated shape (e.g., rectangle or ellipse) and/or a curved shape (e.g., a curvature to match natural curvature of surface of a head) as illustrated. Electrode 150 surfaces may be coated in gold, silver, silver chloride, or other conductive polymer. The texture of electrodes 150 may be smooth or any other texture that can increase a surface area of the electrode.

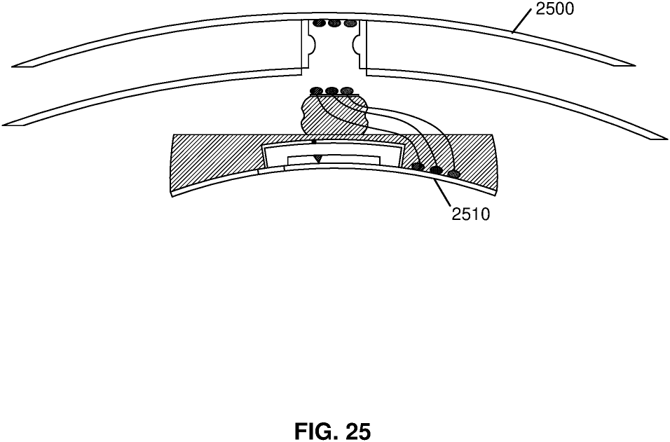

An illustrative process for replacing electrodes is illustrated in FIG. 25. In this illustration, headset 2500 is provided with a replaceable EEG electrode 2510. Headset 2500 and electrode 2510 may be similar to headset 100 and electrode 150 illustrated in FIG. 1. For example, three connection points may be connected to electrode 2510. When electrode 2510 is communicatively coupled with headset 2500, the data signals, power signals, and other communications may be transmitted between electrode 2510 and headset 2500.

In some examples, the connection points may be ports to plug in electrode 2510 into headset 2500. When electrode 2510 becomes inoperable, electrode 2510 may be unplugged from the port of headset 2500 (e.g., each of the three connection points) and replaced with a new electrode for easy replacement.

### 3D Angular Mapping

Headset 100 may be used to collect data using an IMU within the headset as a control system, which may also perform the analysis and processing. The IMU may include an accelerometer, gyroscope, feature extraction, sensor fusion, and other components or engines for generating data. The data can be stored, inferred, or retrieved from a memory incorporated with headset 100 as well. In some examples, the data may be transmitted to a computer system for processing and analysis.

The IMU may be an electronic device incorporated with headset 100 that measures and reports the specific force, angular rate, and orientation of a user’s body (e.g., head). For example, this data may be generated using one or more of an accelerometer, gyroscope, or magnetometer as components of the IMU. In some examples, six degrees of freedom (DOF) may be measured by the IMU, including acceleration, force, and angular velocity acting upon the X, Y, and Z axis.

In some examples, the IMU may be mounted on the motherboard (e.g., PCB board) which will be located on the expanded area extending behind the ear and wrapping around the back of the head. In some examples, the IMU may be mounted on the right side of the headset or the left side of the headset. In some examples, the exact location of the IMU may not matter.

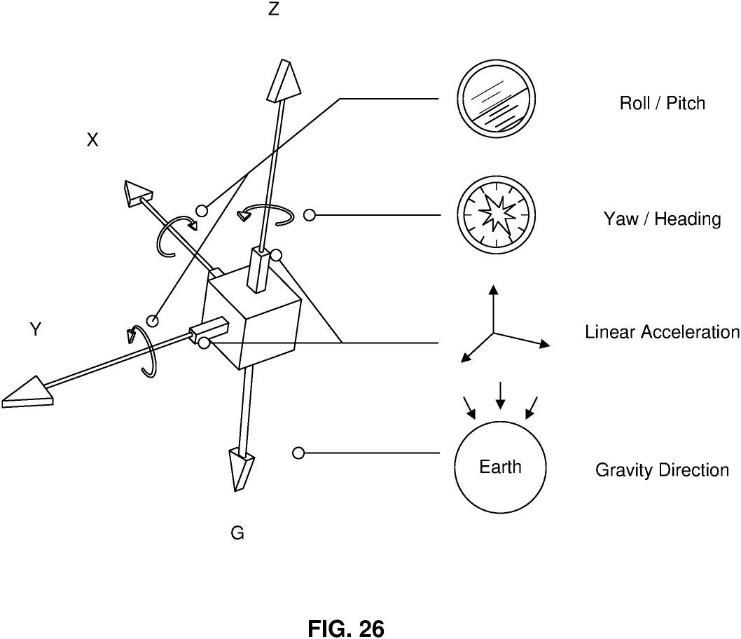

An illustrative example of generating inertial data is provided with FIG. 26 in relation to the X, Y, and Z axis. The data may comprise a roll or pitch along the X-axis, the linear acceleration along the X or Y axis, and the yaw, heading, or gravity direction along the Z-axis.

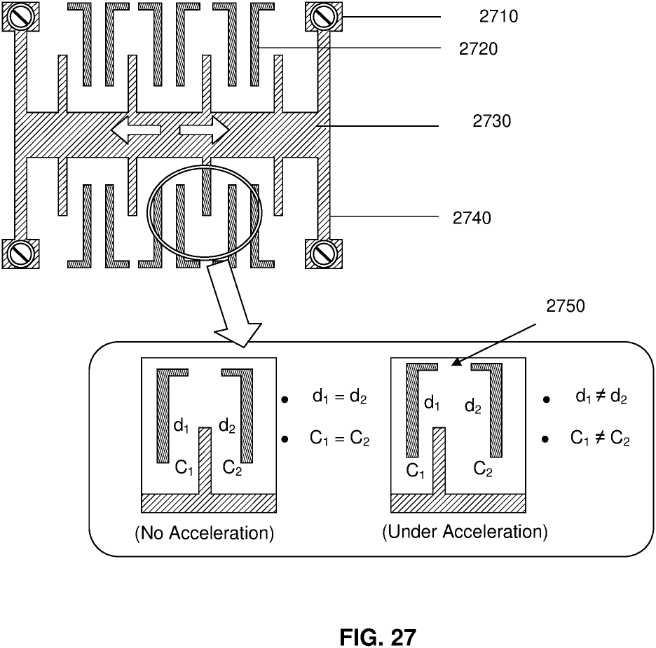

An illustrative example of generating accelerometer data is provided with FIG. 27. In this illustration, one or more electrodes 150 are installed with headset 100 and the headset incorporates an accelerometer. The accelerometer may comprise anchor 2710, fixed electrodes 2720, movable seismic mass 2730, and tether or spring 2740. A differential capacitor pair 2750 is also illustrated to show the relation of the movable seismic mass 2730 to be fixed electrodes 2720 during acceleration.

For example, the accelerometer may measure acceleration (e.g., the rate of change of the velocity of an object). The accelerometer of headset 100 may measure the acceleration in meters per second squared (m/s²) or in G-forces (g) by sensing either static forces (e.g., gravity) or dynamic forces (e.g., vibrations and movement) of acceleration. Additionally, accelerometers are useful for sensing vibrations in systems or for orientation applications.

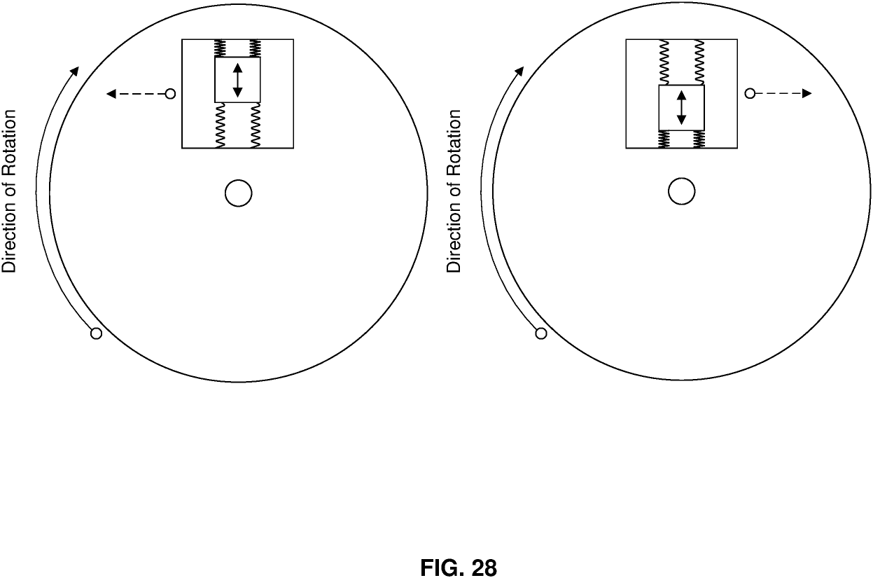

An illustrative example of generating gyroscope data is provided with FIG. 28. The gyroscope of headset 100 may measure rotational motion microelectromechanical system (MEMS) or angular velocity (e.g., in degrees per second) or the rate of change of the angular position over time (angular velocity) with a unit of (deg./s). Gyroscopes are useful for sensing an angle of rotation in systems or for orientation applications.

In some examples, feature extraction may be implemented, including instantaneous force exerted on object (e.g., using accelerometer data) or instantaneous angle of object (e.g., using gyroscope data). For example, an angle of an object can be calculated by integrating gyroscope data over time t. To obtain the angular position, the angular velocity may be integrated with t=0 theta=0. The angular position can be determined at any moment t with the following equation:

$$θ \left(t\right) = {\int }_{0}^{t} \dot{θ} \left(t\right) d t \approx {\sum }_{0}^{t} \dot{θ} \left(t\right) {T}_{s}$$

In some examples, the angle of an object can be calculated from the accelerometer data using the gravity vector (e.g., gravitational force acting upon the sensor at all times). Along with the gravitational force, other systems may act upon object as well and accelerometer data may be filtered to remove high frequency noise (e.g., caused due to vibration or shocks).

In some examples, sensor fusion may be implemented (e.g., to bring together inputs from accelerometer and gyroscope to form a single model). The data may include drift over time due to continuous integration over time and accelerometer data observed at instant time t and/or stable over a long time interval (e.g., using a complementary filter or Kalman filter). The output of the analysis may include a 3D angular position of an object that can be mapped in 3D space using pitch, roll, and yaw values. The analysis may also consider whether the user is stationary or in motion.

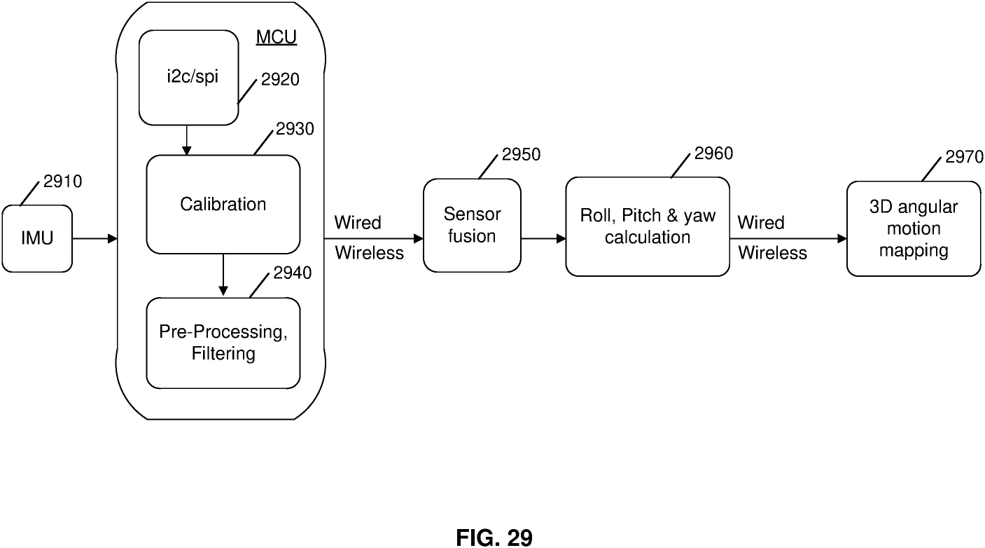

FIG. 29 illustrates 3D angular mapping using various components of the headset, in accordance with one or more implementations of the invention. Headset 100 illustrated in FIG. 1 may implement the process described herein.

At block 2910, the IMU may collect data from an accelerometer, gyroscope, feature extraction, sensor fusion, and other components or engines of the headset.

At block 2920, the microcontroller unit (MCU) may receive sensor information via the inter-integrated circuit (I²C) or serial peripheral interface (SPI).

At block 2930, the data may be calibrated.

At block 2940, the data may be pre-processed and/or filtered.

At block 2950, the data may be provided for sensor fusion to combine the sensory data or data derived from disparate sources. The resulting information may have less uncertainty than would be possible when these sources were used individually.

At block 2960, the roll, pitch, and yaw calculation may be implemented.

At block 2970, the 3D angular motion mapping may be implemented.

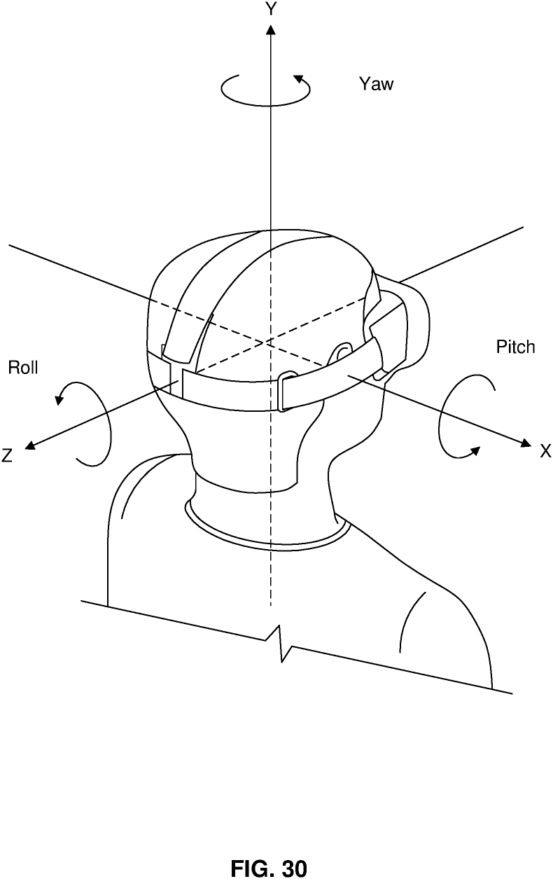

FIG. 30 illustrates the user’s head movement for generating data, in accordance with one or more implementations of the invention. The headset may move along the X, Y, and Z axis. While the user wears the headset 100, the headset may record data along each axis.

### Headset as a Human Computer Interface

Headset 100 may receive the data from the sensors and implement controls based on the data. As an illustrative example, the headset may be moved along an X and Y axis and, on a corresponding graphical user interface provided at a display coupled with the computer system, an object can be moved in accordance with the headset movement. In other words, the headset may be used to control the object using head motion as an alternative human-computer interface device.

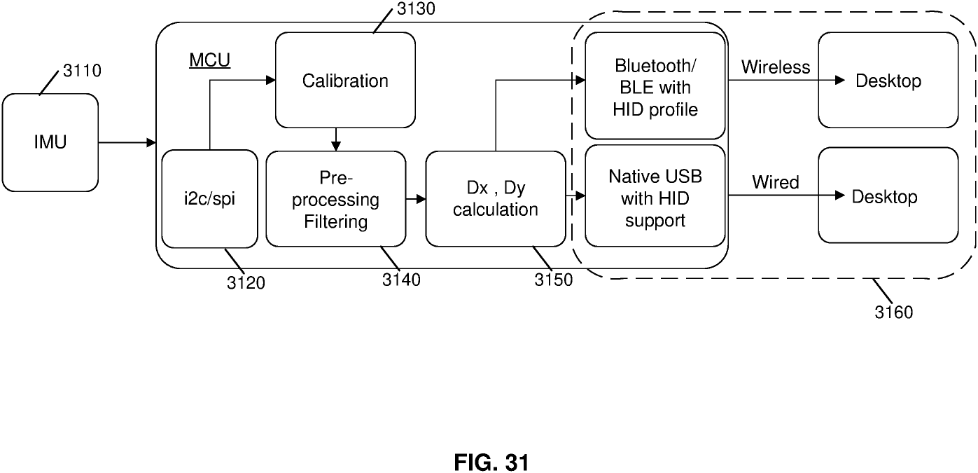

FIG. 31 illustrates a process of converting data to interactions with a desktop display, in accordance with one or more implementations of the invention.

At block 3110, the IMU may collect data from an accelerometer, gyroscope, feature extraction, sensor fusion, and other components or engines of the headset.

At block 3120, the microcontroller unit (MCU) may receive sensor information via the inter-integrated circuit (I²C) or serial peripheral interface (SPI).

At block 3130, the data may be calibrated.

At block 3140, the data may be pre-processed and/or filtered.

At block 3150, the movement of object (e.g., displacement) over 2 axis (e.g., X and Y, or X and Z) is calculated over time interval dt. For example, the derivatives of x, y, or z (e.g., Dx, Dy, and Dz) may hold information of movement of headset 100 in the x, y, or z direction over the time interval dt.

At block 3160, various methods of output may be implemented, including wired or wireless communication protocols. When wireless communication is implemented, the derivative data may be transmitted using the Bluetooth/BLE protocol with HID profile, which is transmitted wirelessly to a client device or desktop computer. When wired communication is implemented, the derivative data may be transmitted using a native USB protocol with HID support, which is transmitted through the wired USB connection to a client device or desktop computer.

### System Visualizations and Architecture

One or more other visualizations may be generated using the techniques described herein. For example, in some implementations, a computer system may utilize the various components and techniques described in U.S. Pat. Application No. 17/411,676, the entirety of which is incorporated by reference.

### Example Processes Performed With the Headset

Headset 100 may perform various processes with the sensor data. for example, headset 100 may implement an optimized adaptive spectrum noise cancellation to remove motion artifacts from PPG data, non-invasive heart rate and respiration rate estimation, adaptive spectrum noise cancellation (ASNC), frequency domain artifact removal, or real-time motion artifact removal.

Headset 100 may perform real-time motion artifact removal as the user is in motion while using headset 100. The motion artifact affects the frequency spectrum especially in heart rate and respiration rate frequency range. To prevent false identification of heart rate and respiration rate, an adaptive spectrum noise cancellation algorithm may be used to remove the motion noise from the spectrum. In order to enable online processing, the process may implement spectrum denoising, heart rate calculation, and respiration rate calculation before the next data is received to a data buffer. Faster calculations can be achieved by reducing the number of iterations required in mathematical calculations.

In PPG, useful information may be identified in very low to low frequency regions (e.g., 0.01 Hz to 10 Hz). Any frequency data after 10 Hz may be removed to achieve a faster execution time and lower use of memory. This may also optimize DCT and Hilbert transform calculations to the required frequency range. By removing the unnecessary data, the process may perform faster than other algorithms to remove the motion activity of the head of the user with noise cancellation.

The first stage may implement digital filters to filter signals out of frequency of interest. Each data may pass through different filters for heart rate, respiration rate, and SpO2 calculations. This filtered data may be transformed to frequency spectrum using Discrete Fourier Transform (DFT). The process may also implement discrete cosine transform (DCT) from a frequency range 0 Hz to 5 Hz for accelerometer data, red PPG data, and IR PPG data. The process may also implement a Hilbert transform function to envelope a noisy PPG signal from 0 Hz to 5 Hz for accelerometer data, red PPG data, and IR PPG data. The process may also implement a Moore-Penrose inverse of accelerometer data. The adaptive gain may be calculated based on the envelope of accelerometer data and IR signal. Using the calculated adaptive gain value, the accelerometer data may be scaled up to match the magnitude of the PPG spectrum. The final cleaning of the motion artifact spectrum from IR PPG spectrum may be implemented to remove false motion induced peaks from PPG spectrum.

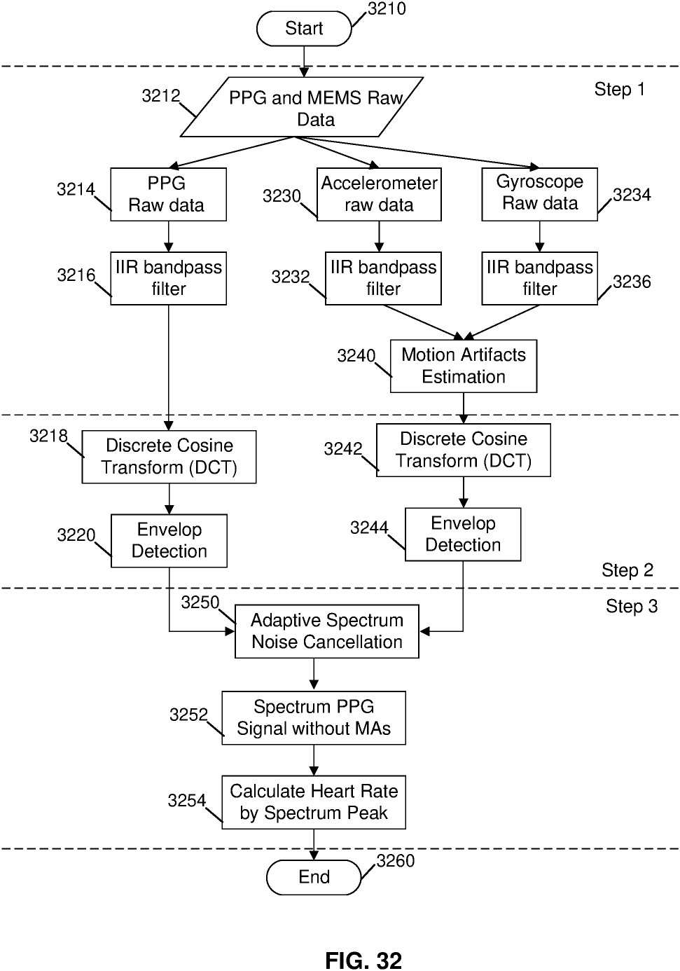

FIG. 32 illustrates an example of a process of calculating a heart rate, in accordance with one or more implementations of the invention.

At block 3210, the process begins.

At block 3212, the process receives the PPG and MEMS raw data.

At block 3214, the process identifies the PPG raw data.

At block 3216, the process provides the PPG raw data to the IIR bandpass filter.

At block 3218, the process provides the data to a discrete cosine transform (DCT).

At block 3220, the process provides the data to the envelop detection.

At block 3230, the process identifies the accelerometer raw data.

At block 3232, the process provides the accelerometer raw data to the IIR bandpass filter.

At block 3234, the process identifies the gyroscope raw data.

At block 3236, the process provides the gyroscope raw data to the IIR bandpass filter.

At block 3240, the process provides the accelerometer data and the gyroscope data to the motion artifacts estimation.

At block 3242, the process provides the data to a discrete cosine transform (DCT).

At block 3244, the process provides the data to the envelop detection.

At block 3250, the process provides the data is provided to the adaptive spectrum noise cancellation.

At block 3252, the process provides the data to a spectrum PPG signal without Mas.

At block 3254, the process calculates a heart rate by spectrum peak.

At block 3260, the process ends.

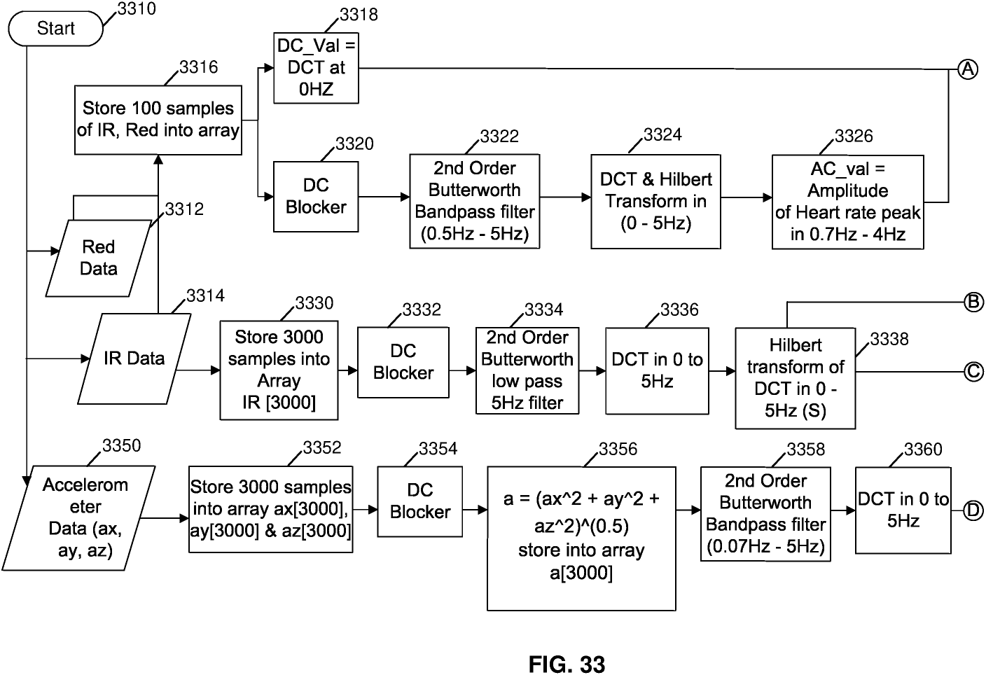

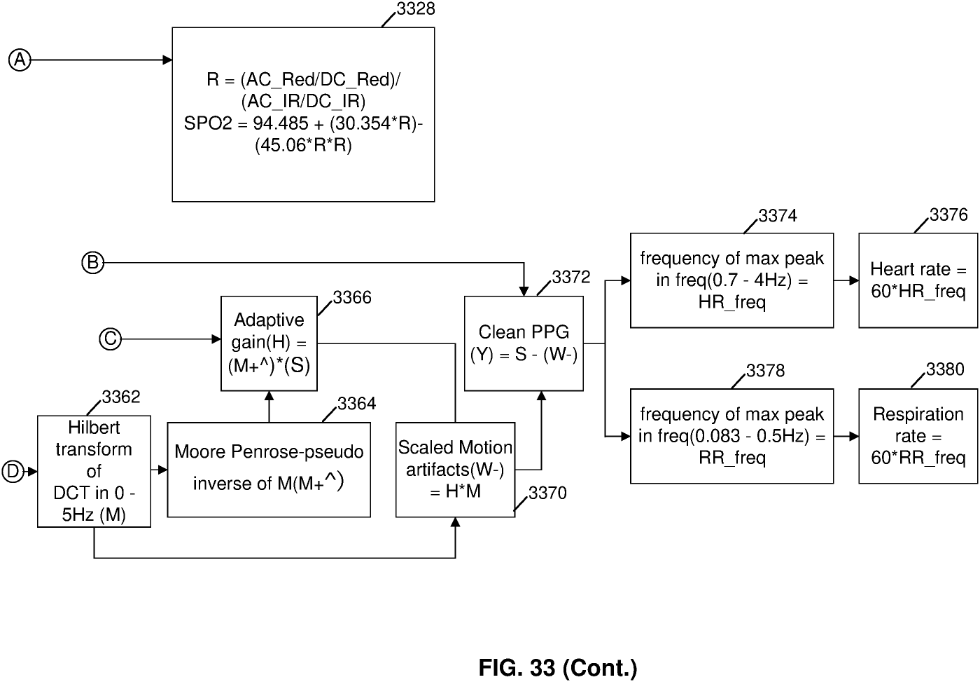

FIG. 33 illustrates an example of a process of calculating a heart rate and/or respiration rate, in accordance with one or more implementations of the invention.

At block 3310, the process begins by receiving red data, IR data, or accelerometer data.

At block 3312, the process identifies the red data.

At block 3314, the process identifies the IR data.

At block 3316, the process stores a plurality of samples (e.g., 100) of IR data and red data into an array.

At block 3318, the process calculates DC_Val = DCT at 0 Hz.

At block 3320, the process implements the DC blocker.

At block 3322, the process determines a second order Butterworth Bandpass filter (e.g., 0.5 Hz - 5 Hz).

At block 3324, the process determines a DCT and Hilbert Transform (e.g., 0 Hz - 5 Hz).

At block 3326, the process calculates AC_Val = Amplitude of heart rate peak (e.g., 0.7 Hz - 4 Hz).

At block 3328, the process calculates R = (AC_Red/DC_Red)/(AC_IR/DC_IR) and SPO2 = 94.485 + (30.354*R)-(45.06*R*R)

At block 3330, the process stores a plurality of samples (e.g., 3,000) of IR data into an array (e.g., IR[3000]).

At block 3332, the process implements the DC blocker.

At block 3334, the process determines a second order Butterworth Bandpass low pass filter (e.g., 5 Hz).

At block 3336, the process determines a DCT (e.g., 0 Hz - 5 Hz).

At block 3338, the process determines a Hilbert Transform of DCT (e.g., 0 Hz - 5 Hz).

At block 3350, the process identifies the accelerometer data.

At block 3352, the process stores a plurality of samples (e.g., 3,000) of accelerometer data into an array (e.g., ax[3000], ay[3000], and az[3000]).

At block 3354, the process implements the DC blocker.

At block 3356, the process calculates a = (ax^2 + ay^2 + az^2)^(0.5). The result may be stored into array a[3000].

At block 3358, the process determines a second order Butterworth Bandpass filter (e.g., 0.07 Hz - 5 Hz).

At block 3360, the process determines a DCT (e.g., 0 Hz - 5 Hz).

At block 3362, the process determines a Hilbert Transform of DCT (e.g., 0 Hz - 5 Hz).

At block 3364, the process implements Moore Penrose-pseudo inverse of M(M+^).

At block 3366, the process implements Adaptive gain(H) = (M+^)*(S).

At block 3370, the process implements Scaled Motion artifacts (W-) = H*M.

At block 3372, the process implements Clean PPG (Y) = S - (W-).

At block 3374, the process calculates the frequency of max peak in freq(0.7 - 4 Hz) = HR_freq.

At block 3376, the process calculates the heart rate as 60 * HR_freq.

At block 3378, the process calculates the frequency of max peak in freq(0.083 - 5 Hz) = RR_freq.

At block 3380, the process calculates the respiration rate as 60 * RR_freq.

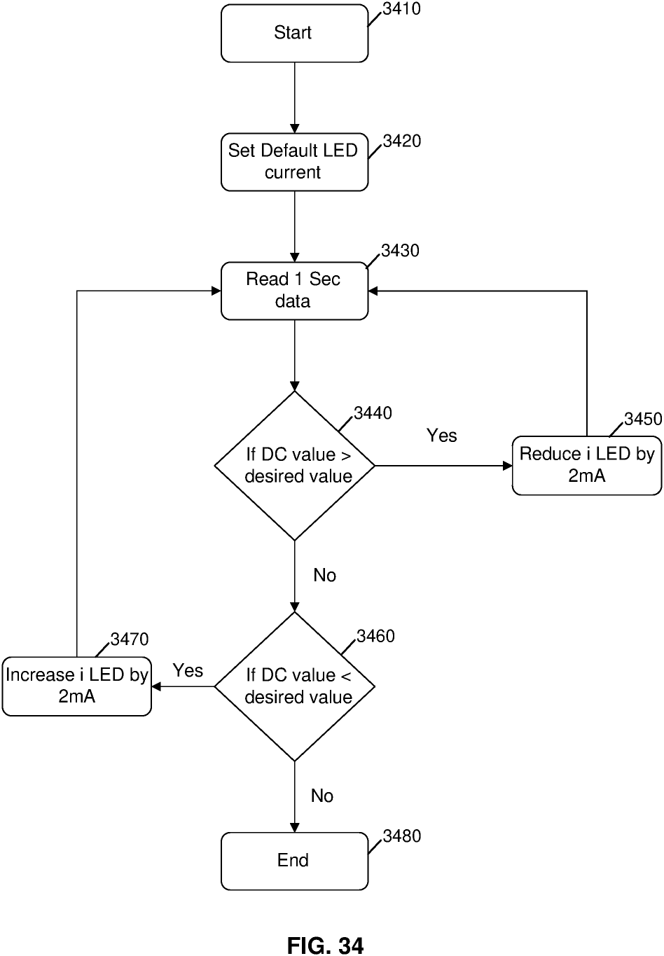

FIG. 34 illustrates an example of a process of determining LED current, in accordance with one or more implementations of the invention.

At block 3410, the process begins.

At block 3420, the process sets a default LED current

At block 3430, the process determines a subset of data (e.g., one second of data).

At block 3440, if the DC value is greater than a desired value, the process proceeds to block 3450. If not, the process proceeds to block 3460.

At block 3450, the process reduces i LED by 2 mA.

At block 3460, if the DC value is less than a desired value, the process proceeds to block 3470. If not, the process proceeds to block 3480.

At block 3470, the process increases i LED by 2 mA.

At block 3480, the process ends.

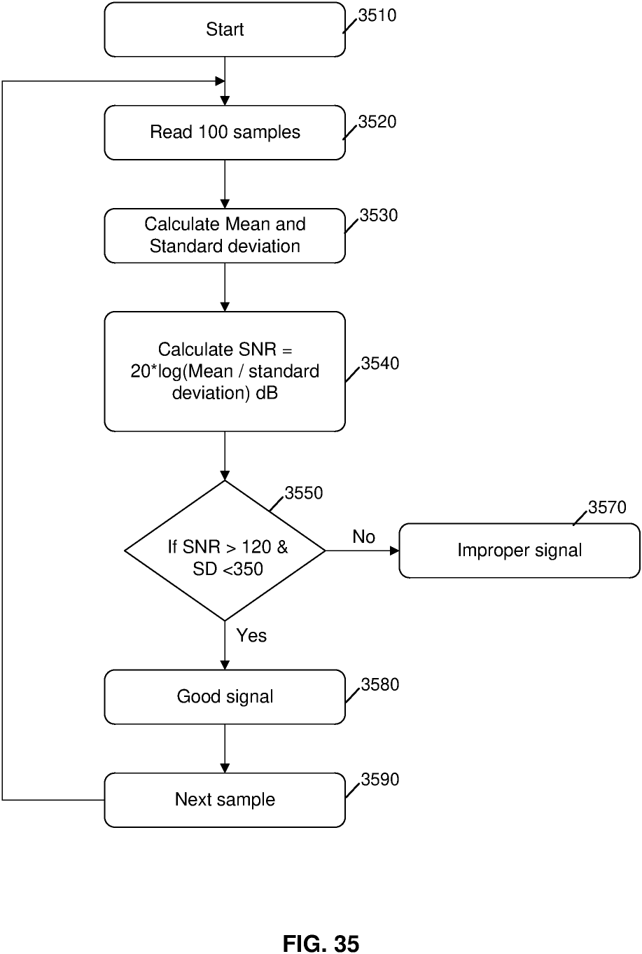

FIG. 35 illustrates an example of a process of determining a good or improper signal, in accordance with one or more implementations of the invention.

At block 3510, the process begins

At block 3520, the process receives a plurality of sample data (e.g., 100 data samples).

At block 3530, the process calculates the mean and standard deviations.

At block 3540, the process calculates the SNR as 20*log(Mean / standard deviation) dB.

At block 3550, if the SNR value is greater than 120 and SD is less than 350, the process proceeds to block 3580. If not, the process proceeds to block 3570.

At block 3570, the process determines an improper signal.

At block 3580, the process determines a good signal.

At block 3590, the process receives a next sample and proceeds back to block 3510.

For purposes of explanation, numerous specific details are set forth in order to provide a thorough understanding of the description. It will be appreciated by those having skill in the art that the implementations described herein may be practiced without these specific details or with an equivalent arrangement. Accordingly, it is to be understood that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

Implementations of the disclosure may be made in hardware, firmware, software, or any suitable combination thereof. Aspects of the disclosure may be implemented as instructions stored on a machine-readable medium, which may be read and executed by one or more processors. A machine-readable medium may include any mechanism for storing or transmitting information in a form readable by a machine (e.g., a computing device). For example, a tangible computer readable storage medium may include read only memory, random access memory, magnetic disk storage media, optical storage media, flash memory devices, and others, and a machine-readable transmission media may include forms of propagated signals, such as carrier waves, infrared signals, digital signals, and others. Firmware, software, routines, or instructions may be described herein in terms of specific exemplary aspects and implementations of the disclosure, and performing certain actions.

The various instructions described herein are exemplary only. Other configurations and numbers of instructions may be used, so long as the processor(s) are programmed to perform the functions described herein. The description of the functionality provided by the different instructions described herein is for illustrative purposes, and is not intended to be limiting, as any of instructions may provide more or less functionality than is described. For example, one or more of the instructions may be eliminated, and some or all of its functionality may be provided by other ones of the instructions.

In some implementations, the headset may comprise one or more processing units. These processing units may be physically located within the same device. In some implementations, one or more processors may be implemented by a cloud of computing platforms operating together as one or more processors. Processor(s) be configured to execute one or more components by software; hardware; firmware; some combination of software, hardware, and/or firmware; and/or other mechanisms for configuring processing capabilities on processor(s). As used herein, the term “component” may refer to any component or set of components that perform the functionality attributed to the component. This may include one or more physical processors during execution of processor readable instructions, the processor readable instructions, circuitry, hardware, storage media, or any other components. Furthermore, it should be appreciated that various instructions may be executed locally or remotely from the other instructions.

The various instructions described herein may be stored in a storage device, which may comprise random access memory (RAM), read only memory (ROM), and/or other memory. For example, one or more storage devices may comprise any tangible computer readable storage medium, including random access memory, read only memory, magnetic disk storage media, optical storage media, flash memory devices, and/or other memory configured to computer-program instructions. In various implementations, one or more storage device may be configured to store the computer program instructions (e.g., the aforementioned instructions) to be executed by the processors as well as data that may be manipulated by the processors. The storage device may comprise floppy disks, hard disks, optical disks, tapes, or other storage media for storing computer-executable instructions and/or data.

One or more databases may be stored in one or more storage devices. The databases described herein may be, include, or interface to, for example, an Oracle™ relational database sold commercially by Oracle Corporation. Other databases, such as Informix™, DB2 (Database 2) or other data storage, including file-based, or query formats, platforms, or resources such as OLAP (On Line Analytical Processing), SQL (Structured Query Language), a SAN (storage area network), Microsoft Access™ or others may also be used, incorporated, or accessed. The database may comprise one or more such databases that reside in one or more physical devices and in one or more physical locations. The database may store a plurality of types of data and/or files and associated data or file descriptions, administrative information, or any other data.

The various components illustrated throughout the disclosure may be coupled to at least one other component via a network, which may include any one or more of, for instance, the Internet, an intranet, a PAN (Personal Area Network), a LAN (Local Area Network), a WAN (Wide Area Network), a SAN (Storage Area Network), a MAN (Metropolitan Area Network), a wireless network, a cellular communications network, a Public Switched Telephone Network, and/or other network. Furthermore, according to various implementations, the components described herein may be implemented in hardware and/or software that configure hardware.

In some instances, well-known structures and devices are shown in block diagram form in order to avoid unnecessarily obscuring the description. In other instances, functional block diagrams and flow diagrams are shown to represent data and logic flows. The components of block diagrams and flow diagrams (e.g., modules, blocks, structures, devices, features, etc.) may be variously combined, separated, removed, reordered, and replaced in a manner other than as expressly described and depicted herein.

Although embodiments are described above with reference to replaceable EEG sensor(s), the EEG sensor(s) described in any of the above embodiments may alternatively be another type of EEG sensor such as a fixed EEG sensor, removable EEG sensor, disposable EEG sensor, etc. Similarly, the PPG sensors and the electrodes described in any of the above embodiments may be replaceable, fixed, removable, and/or disposable. Such alternatives are considered to be within the spirit and scope of the present invention, and may therefore utilize the advantages of the configurations and embodiments described above.

In addition, although embodiments are described above with reference to a frontal piece including two separated adjustable slider arms 110A, 110B, the frontal piece described in any of the above embodiments may alternatively be an adjustable single frontal piece 3610 coupled between opposite side portions of the curved frame, wherein the PPG sensor 3640 and two EEG sensors (3630) along with a bias sensor 3631 (which acts as ground, for example) are positioned along the single frontal piece 3610, as illustrated in FIG. 36. FIG. 36 also illustrates an optional elastic member 3690 that couples the two posterior parts 3608a, 3608b of the posterior curved frame portion together. The elastic member 3690 aids in maintaining adequate tension of the headset across the head so there is sufficient pressure on the electrodes against the head to improve signal quality. The two posterior parts may further optionally be separated with a gap (i.e., without a connection or coupling to each other) when worn by the user, as illustrated in FIG. 4. As a yet further option, the two posterior parts may be a unitary structure. Such alternatives are considered to be within the spirit and scope of the present invention, and may therefore utilize the advantages of the configurations and embodiments described above.

Further, although embodiments are described above with reference to sensors 140, 3640 being PPG sensors, any or all the PPG sensors described in any of the above embodiments may alternatively be EEG sensors (and can utilize the locations of those PPG sensors). Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

Reference in this specification to “one implementation”, “an implementation”, “some implementations”, “various implementations”, “certain implementations”, “other implementations”, “one series of implementations”, or the like means that a particular feature, design, structure, or characteristic described in connection with the implementation is included in at least one implementation of the disclosure. The appearances of, for example, the phrase “in one implementation” or “in an implementation” in various places in the specification are not necessarily all referring to the same implementation, nor are separate or alternative implementations mutually exclusive of other implementations. Moreover, whether or not there is express reference to an “implementation” or the like, various features are described, which may be variously combined and included in some implementations, but also variously omitted in other implementations. Similarly, various features are described that may be preferences or requirements for some implementations, but not other implementations.

The language used herein has been principally selected for readability and instructional purposes, and it may not have been selected to delineate or circumscribe the inventive subject matter. Other implementations, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A headset comprising:
a curved frame comprising:
a frontal curved frame portion configured to be worn on a forehead portion of a head of a user; and
an upper curved frame portion configured to be worn on an upper head portion of the head of the user;
one or more PPG sensors coupled to the frontal curved frame portion; and
one or more EEG sensors coupled to the frontal curved frame portion and/or upper curved frame portion.

2. The headset of claim 1, wherein the one or more additional EEG sensors are coupled to only the upper curved frame portion.

3. The headset of claim 1, wherein each of the EEG sensors comprises a curved outer surface, and wherein the coupling location of each EEG sensor to the frontal curved frame portion and/or upper curved frame portion and a shape of the curved outer surface of each EEG sensor are configured to correspond with a corresponding location and curvature of the upper head portion of the head of the user.

4. The headset of claim 1, wherein each of the EEG sensors is a capacitive non-dermal contact type EEG sensor that is configured to be positioned either in direct contact with skin or be positioned over hair on the head of the user, when the headset is worn by the user.

5. The headset of claim 1, wherein the front curved frame portion comprises two length-adjustable slider arms that are retractable and extendable from opposite side portions of the curved frame.

6. The headset of claim 1, wherein the frontal curved frame portion comprises an adjustable single frontal piece coupled between opposite side portions of the curved frame, and wherein the PPG and EEG sensors are positioned along the single frontal piece.

7. The headset of claim 1, wherein the curved frame further comprises a posterior curved frame portion, and wherein one or more additional EEG sensors are coupled to the posterior curved frame portion.

8. The headset of claim 1, wherein the curved frame further comprises a posterior curved frame portion comprising two posterior parts.

9. The headset of claim 8, wherein the headset further comprises one or more electrodes coupled to at least one of the two posterior parts.

10. The headset of claim 9, wherein the two posterior parts are coupled together via an elastic member.

11. The headset of claim 1, wherein each of the EEG sensors is removable or replaceable.

12. The headset of claim 1, wherein the headset further comprises a charging port.

13. The headset of claim 1, wherein the headset further comprises one or more LED lights indicating power, charging status, and/or connection to a host device.

14. The headset of claim 1, wherein the headset further comprises an IMU sensor.

15. A method for measuring EEG signals using a headset to detect brain activity, wherein the headset comprises or is coupled to a computer system comprising one or more processors programmed with computer program instructions that, when executed by the one or more processors, cause the computer system to perform a method, wherein the method comprises:
receiving, by one or more sensors, raw EEG data; and
applying spectral analysis techniques to one or more channels of the raw EEG data to isolate spectral components in the channels.

16. The method of claim 15, wherein the method further comprises:
combining the raw EEG data with concurrent photoplethysmography (PPG) data and inertial measurement unit (IMU) data; and
applying biometric analysis and/or classification to the PPG data and IMU data.

17. The method of claim 15, wherein the method further comprises moving an object on a display of the computer system using input data from an inertial measurement unit (IMU) sensor.

18. The method of claim 15, wherein the headset further comprises:
a curved frame comprising:
a frontal curved frame portion configured to be worn on the forehead portion of the head of the user; and
an upper curved frame portion configured to be worn on an upper head portion of the head of the user;
one or more PPG sensors coupled to the frontal curved frame portion; and
one or more EEG sensors coupled to the frontal curved frame portion and/or upper curved frame portion.

19. The method of claim 18, wherein the one or more additional EEG sensors are coupled to only the upper curved frame portion.

20. The method of claim 18, wherein each of the EEG sensors comprises a curved outer surface, and wherein the coupling location of each EEG sensor to the frontal curved frame portion and/or upper curved frame portion and a shape of the curved outer surface of each EEG sensor are configured to correspond with a corresponding location and curvature of the upper head portion of the head of the user.

21. The method of claim 18, wherein each of the EEG sensors is a capacitive non-dermal contact type EEG sensor that is configured to be positioned either in direct contact with skin or be positioned over hair on the head of the user, when the headset is worn by the user.

22. The method of claim 18, wherein the frontal curved frame portion comprises an adjustable single frontal piece coupled between opposite side portions of the curved frame, and wherein the PPG and EEG sensors are positioned along the single frontal piece.

23. The method of claim 18, wherein the curved frame further comprises a posterior curved frame portion, and wherein one or more additional EEG sensors are coupled to the posterior curved frame portion.

24. The method of claim 18, wherein the curved frame further comprises a posterior curved frame portion comprising two posterior parts, wherein the headset further comprises one or more electrodes coupled to at least one of the two posterior parts.

25. The method of claim 24, wherein the two posterior parts are coupled together via an elastic member.
